(19) United States
(12) Patent Application Publication
(10) Pub. No.: US 2021/0148913 A1
(43) Pub. Date: May 20, 2021

(54) ELECTROCHEMICAL SENSOR FOR ANALYTE DETECTION

(71) Applicant: CASE WESTERN RESERVE UNIVERSITY, Cleveland, OH (US)

(72) Inventors: Chung Chiun Liu, Cleveland Heights, OH (US); Yifan Dai, Cleveland, OH (US); Jiwei Yao, Cleveland, OH (US); Yuan Wang, Cleveland, OH (US)

(21) Appl. No.: 17/047,347

(22) PCT Filed: Apr. 15, 2019
(86) PCT No.: PCT/US2019/027517

§ 371 Date: Oct. 13, 2020

Related U.S. Application Data
(60) Provisional application No. 62/657,326, filed on Apr. 13, 2018.

(51) Int. Cl. G01N 33/574; G01N 33/543

(52) U.S. Cl. CPC G01N 33/57434; G01N 33/5438

(57) ABSTRACT

A sensor for the detection of AMACR and/or PSA includes a substrate, a working electrode and counter electrode formed on a surface of the substrate, and an anti-AMACR antibody and/or anti-PSA antibody bioconjugated to a surface of an exposed portion of the working electrode.

### RELATED APPLICATION

This application claims priority from U.S. Provisional Application Nos. 62/657,326, filed Apr. 13, 2018, the subject matter of which is incorporated herein by reference in its entirety.

### BACKGROUND

Prostate cancer was the most prevalent cancer in men for new cancer diagnoses and the third leading cause of cancer death in the United States in 2017. Metabolic syndrome was considered as a possible risk factor, and there was insufficient evidence for the correlation. Prostate-specific antigen (PSA) is a widely used biomarker in clinical screening for prostate cancer. However, PSA also acted as a biomarker in many noncancerous conditions, such as inflammation, infection, trauma, and benign prostatic hyperplasia. Thus, the screening of PSA led to poor specificity, especially with an intermediate range of valid PSA concentration of 4-10 ng mL⁻¹. Therefore, a more specific biomarker is desirable for early diagnosis of prostate cancer. One of the emerging biomarkers for prostate cancer is alpha-methylacyl-CoA racemase (AMACR), a metabolic enzyme, which has been proven to be a highly expressed biomarker in prostate cancer cells. AMACR, a peroxisomal enzyme, facilitates β-oxidation of branched-chain fatty acids by changing (2R,S,6R,10R)-pristanoyl-CoA to (2S,S,6R,10R)-pristanoyl-CoA. The mutations in the gene AMACR lead to reduced enzyme activity caused by the raised level of branched-chain fatty acids. Messenger-RNA(mRNA) of AMACR is overexpressed in prostate epithelium during the translation process. For prostate cancer patients, the mRNA level of AMACR was around 9 times higher compared to that of the control group, and the protein AMACR level increased around 2.5 times comparing to that of the normal group, making AMACR an ideal biomarker for prostate cancer. Although AMACR is a tissue-carried protein, studies proved the possibilities of detecting AMACR directly from body fluid samples of prostate cancer patients.

Conventional methods for the detection of AMACR included techniques of enzyme-linked immunosorbent assay, radioimmunoassay, chemiluminescence immunoassay, and fluoro-immunoassay (HA). The FIA method showed that the detection limit of AMACR was down to 4.6 pg mL⁻¹. The accuracy of detection using these techniques was applicable for screening purposes. However, these tests were laborious, time-consuming, and expensive.

### SUMMARY

Embodiments described herein relate to a detection system, method, and in vitro assay for detecting, identifying, quantifying, and/or determining the levels of AMACR and/or PSA levels in a bodily sample as well as to a detection system, method, and in vitro assay for diagnosing, identifying, staging, and/or monitoring cancer in a subject having or suspected of having cancer.

In some embodiments, the system and/or method for detecting, indentifying, quantifying, and/or determining cancer (e.g., prostate cancer) can detect, indentify, quantify, and/or determine the amount or level of AMACR and/or PSA in a sample. The system can include an electrochemical biosensor, for detecting, identifying, quantifying, and/or determining the amount or level of AMACR and/or PSA in a sample, such as blood. The system and method described herein can provide a single use, disposable, and cost-effective means for simple assessment of AMACR and/or PSA in biological samples obtained by non-invasive or minimally invasive means.

In some embodiments, the system and methods described herein include an electrochemical biosensor, a redox solution, and a measuring device. The electrochemical biosensor can produce a signal that is related to the presence or quantity of the AMACR and/or PSA being detected in a sample. In some embodiments, the system can be used to detect and/or quantify AMACR and/or PSA that is present in blood or a biological fluid.

In some embodiments, the biosensor includes a substrate, a working electrode formed on a surface of the substrate, a counter electrode formed on the surface of the substrate, and a dielectric layer covering a portion of the working electrode and counter electrode and defining an aperture exposing other portions of the working electrode and counter electrode. A plurality of bioconjugated anti-AMACR antibodies and/or anti-PSA antibodies that are selective to AMACR or PSA, respectively, are conjugated to a surface of the exposed portion of the working electrode. Each of the bioconjugated anti-AMACR antibodies and/or anti-PSA antibodies can include a linker that conjugates the bioconjugated anti-AMACR antibodies and/or anti-PSA antibodies to the surface of the working electrode. The linker can be about 3 to about 6 atoms in length and include a first end and a second end. The first end can include an acyl group that is bound to a lysine group of the anti-AMACR and/or anti-PSA antibody and the second end can include a sulfhydryl group that is bound to the surface of the working electrode. The surface of the working electrode is free of a self-assembled monolayer.

The redox solution is applied to the working electrode for determining the quantity of AMACR and/or PSA in the sample bound to the anti-AMACR antibodies and/or anti-PSA antibodies. The measuring device applies voltage potentials to the working electrode and counter electrode and measures the current flow between the working electrode and counter electrode to determine the level of the AMACR and/or PSA in a sample, such as blood. In some embodiments, the interaction of anti-AMACR antibodies and/or anti-PSA antibodies and the bound AMACR and/or PSA can be detected using electrochemical analytical techniques, such as cyclic voltammetry (CV), differential pulse voltammetry (DPV), to determine the presence of the AMACR and/or PSA in the biological sample.

In some embodiments, the working electrode and the counter electrode include metalized films. The metalized films used to form the working electrode and the counter electrode can independently comprise gold, platinum, palladium, silver, alloys thereof, and composites thereof. The metalized films can be provided on the surface of the substrate by sputtering or coating the films on the surface and then laser ablating the films to form the working electrode and counter electrode.

In other embodiments, the sensor can include a reference electrode on the surface of the substrate. The dielectric can cover a portion of the reference electrode.

Other embodiments described herein relate to a method of forming an electrochemical sensor for detection of AMACR and/or PSA in a bodily sample to detect cancer (e.g., prostate cancer) in a subject. The method includes providing anti-AMACR antibodies and/or anti-PSA antibodies that are selective to the AMACR and/or PSA, respectively. Heterobifunctional linkers are conjugated to the anti-AMACR antibodies and/or anti-PSA antibodies. The linkers include an acyl group and a sulfhydryl group. A sensor is provided that includes a substrate, a working electrode formed on a surface of the substrate, a counter electrode formed on the surface of the substrate, and an optional reference electrode formed on the surface of the substrate. A dielectric layer covers a portion of the working electrode and counter electrode and defines an aperture exposing portions of surfaces of the working electrode and counter electrode. The anti-AMACR antibodies and/or anti-PSA antibodies conjugated to the linkers are reacted with the surface of the exposed portion of the working electrode to link anti-AMACR antibodies and/or anti-PSA antibodies to the working electrode.

In some embodiments, each linker is conjugated to a lysine residue of anti-AMACR antibody and/or anti-PSA antibody. The linker also includes a sulfhydryl group that reacts with the surface of the working electrode. For example, the linker can include an N-hydroxysuccinimide ester that reacts with an amine group of a lysine residue and a sulfhydryl group that reacts with the surface of the working electrode. The sulfhydryl group can also be coupled to a protecting group that is removed prior to reaction of the sulfhydryl group with the surface of the exposed portion of the working electrode. In some embodiments, the linker include at least one of N-succinimidyl S-acetylthioacetate or N-succinimidyl S-acetylthiopropionate.

In some embodiments, the working electrode and the counter electrode include metalized films, such as gold, platinum, palladium, silver, alloys thereof, and composites thereof. The metalized films can be provided on the surface of the substrate by sputtering or coating the films on the surface. The working electrode and the counter electrode can be formed using laser ablation to define the dimensions of the working electrode and the counter electrode. The surface of the working electrode can be free of a self-assembled monolayer.

In other embodiments, the linked anti-AMACR antibodies and/or anti-PSA antibodies can be functionalized to the surface of the working electrode by providing the anti-AMACR antibodies and/or anti-PSA antibodies in a solution that is continuously flowed over the surface of the electrode.

### BRIEF DESCRIPTION OF THE DRAWINGS

FIG. 1 is a schematic illustration of a biosensor in accordance with an aspect of the application.

FIGS. 2(A-B) illustrates (A) Formation of the SAM for biosensor fabrication. (B) Formation of the bioconjugated antibody for biosensor fabrication.

FIGS. 3(A-D) illustrate (A) Sensitivity comparison of four different SAM systems. (B) EIS measurements of the bare electrode (pink), SATA-linker (gray), unconjugated antibody, and SATA-conjugated antibody. (C) CV characterization of the stability of the bioconjugation mechanism based biosensor based on different scan rates ranging from 30 to 100 mV/s. (D) Linear calibration curve of the CV current outputs against the square root of scan rates.

FIGS. 4(A-B) illustrate (A) Topography graph of a bare gold electrode. (B) Topography graph of an antibody film-covered gold electrode.

FIGS. 5(A-D) (A) DPV measurements of the AMACR antigen in 0.1 M PBS. (B) DPV measurements of the AMACR antigen in undiluted human serum. (C) Calibration curve based on the DPV measurement of the AMACR antigen in 0.1 M PBS. (D) Interference test on the AMACR biosensor using PSA antigen.

FIGS. 6(A-D) illustrate (A) DPV measurements of the PSA antigen in 0.1 M PBS. (B) DPV measurements of the PSA antigen in undiluted human serum. (C) Calibration curve based on the DPV measurement of the PSA antigen in undiluted human serum. (D) Interference test on the PSA biosensor using the AMACR antigen.

FIGS. 7(A-D) illustrate A) Impedance difference before and after covering of antibody based on 1 mM 3-Mercaptopropionic acid (3-MPA) monolayer. B) Impedance difference before and after covering of antibody based on 5 mM 11-Mercaptoundecanoic acid (MUA). C) Impedance difference before and after covering of antibody based on 1 mM MUA and 10 mM 3-MPA. D) Impedance difference before and after covering of antibody based on 3 mM MUA and 9 mM MCH.

### DETAILED DESCRIPTION

Unless specifically addressed herein, all terms used have the same meaning as would be understood by those of skilled in the art of the subject matter of the application. The following definitions will provide clarity with respect to the terms used in the specification and claims.

As used herein, the term “monitoring” refers to the use of results generated from datasets to provide useful information about an individual or an individual's health or disease status. “Monitoring” can include, for example, determination of prognosis, risk-stratification, selection of drug therapy, assessment of ongoing drug therapy, determination of effectiveness of treatment, prediction of outcomes, determination of response to therapy, diagnosis of a disease or disease complication, following of progression of a disease or providing any information relating to a patient's health status over time, selecting patients most likely to benefit from experimental therapies with known molecular mechanisms of action, selecting patients most likely to benefit from approved drugs with known molecular mechanisms where that mechanism may be important in a small subset of a disease for which the medication may not have a label, screening a patient population to help decide on a more invasive/expensive test, for example, a cascade of tests from a non-invasive blood test to a more invasive option such as biopsy, or testing to assess side effects of drugs used to treat another indication.

As used herein, the term “quantitative data” or “quantitative level” or “quantitative amount” refers to data, levels, or amounts associated with any dataset components (e.g., markers, clinical indicia,) that can be assigned a numerical value.

As used herein, the term “subject” refers to a human or another mammal. Typically, the terms “subject” and “patient” are used herein interchangeably in reference to a human individual.

As used herein, the term “bodily sample” refers to a sample that may be obtained from a subject (e.g., a human) or from components (e.g., tissues) of a subject. The sample may be of any biological tissue or fluid with, which analytes described herein may be assayed. Frequently, the sample will be a “clinical sample”, i.e., a sample derived from a patient. Such samples include, but are not limited to, bodily fluids, e.g., saliva, breath, urine, blood, plasma, or sera; and archival samples with known diagnosis, treatment and/or outcome history. The term biological sample also encompasses any material derived by processing the bodily sample. Processing of the bodily sample may involve one or more of, filtration, distillation, extraction, concentration, inactivation of interfering components, addition of reagents, and the like.

As used herein, the terms “normal” and “healthy” are used interchangeably. They refer to an individual or group of individuals who have not shown any symptoms of a disease, condition, or pathology to be detected, and have not been diagnosed with the disease, condition, or pathology. Preferably, the normal individual (or group of individuals) is not on medication. In certain embodiments, normal individuals have similar sex, age, body mass index as compared with the individual from, which the sample to be tested was obtained. The term “normal” is also used herein to qualify a sample isolated from a healthy individual.

As used herein, the terms “control” or “control sample” refer to one or more biological samples isolated from an individual or group of individuals that are normal (i.e., healthy). The term “control”, “control value” or “control sample” can also refer to the compilation of data derived from samples of one or more individuals classified as normal.

As used herein, the term “indicative of cancer”, when applied to an amount of AMACR and/or PSA in a bodily sample, refers to a level or an amount, which is diagnostic of a cancer (e.g., prostate cancer) such that the level or amount is found significantly more often in subjects with the cancer than in subjects without the cancer (as determined using routine statistical methods setting confidence levels at a minimum of 95%). Preferably, a level or amount, which is indicative of a cancer, is found in at least about 60% of subjects who have the cancer and is found in less than about 10% of subjects who do not have the cancer. More preferably, a level or amount, which is indicative of a cancer is found in at least about 70%, at least about 75%, at least about 80%, at least about 85%, at least about 90%, at least about 95% or more in subjects who have the cancer and is found in less than about 10%, less than about 8%, less than about 5%, less than about 2.5%, or less than about 1% of subjects who do not have the cancer.

Embodiments described herein relate to a detection system, method, and in vitro assay for detecting, identifying, quantifying, and/or determining the levels of AMACR and/or PSA in a bodily sample, as well as to a detection system, method, and in vitro assay for diagnosing, identifying, staging, and/or monitoring a prostate cancer in a subject having or suspected of having prostate cancer.

In some embodiments, the system and/or method for detecting, indentifying, quantifying, and/or determining the amounts, levels, and/or concentration of AMACR and/or PSA in a bodily sample as well as diagnosing, identifying, staging, and/or monitoring a prostate cancer in a subject having or suspected of having prostate cancer can include an electrochemical biosensor, for detecting, identifying, quantifying, and/or determining the amount or level of AMACR and/or PSA in a sample, such as blood. The system and method described herein can provide a single use, disposable, and cost-effective means for simple assessment of AMACR and/or PSA in biological samples obtained by non-invasive or minimally invasive means.

In some embodiments, the electrochemical biosensor includes a substrate, a working electrode formed on a surface of the substrate and a counter electrode formed on the surface of the substrate. A dielectric layer covers a portion of the working electrode and counter electrode and defines an aperture exposing other portions of the working electrode and counter electrode. A bioconjugated anti-AMACR antibody and/or anti-PSA antibody is conjugated or linked to a surface of the exposed portion of the working electrode. The bioconjugated anti-AMACR antibody and anti-PSA antibody selectively binds to AMACR and PSA, respectively, in a sample, and the AMACR and/or PSA once bound is detectable by measuring the current flow between the working electrode and counter electrode.

A redox solution can be applied to the working electrode for determining the quantity of AMACR and/or PSA in the sample. A measuring device applies voltage potentials to the working electrode and counter electrode and measures the current flow between the working electrode and counter electrode to determine the level of AMACR and/or PSA in a bodily sample, such as a blood.

In some embodiments, the bio-recognition mechanism of this sensor can be based on the influence of the redox coupling reaction of the redox solution, such as a potassium ferrocyanide/potassium ferricyanide (K₃Fe(CN)₆/K₄Fe(CN)₆) solution, by AMACR and/or PSA and its receptor (anti-AMACR antibody and/or anti-PSA antibody). In the detection of AMACR and/or PSA, the anti-AMACR antibody and/or anti-PSA antibody is used to provide a lock-and-key bio-recognition mechanism. The AMACR and/or PSA interacts with the anti-AMACR antibody and/or anti-PSA antibody, respectively, affecting the electron charge transfer and can influence a redox coupling reaction in the redox solution applied to the working electrode. The level of AMACR and/or PSA bound to the anti-AMACR antibody and/or anti-PSA antibody can be determined by measuring current flow between the working and counter electrode to which the sample and redox solution has been applied and comparing the measured current to a control value, which can be based on a measured current between the working electrode and counter electrode that is free of bound anti-AMACR antibody and/or anti-PSA antibody.

Differential pulse voltammetry (DPV) can employed as the transduction mechanism of this biosensor to determine the level of bound AMACR and/or PSA. DPV applies a linear sweep voltammetry with a series of regular voltage pulses superimposed on the linear potential sweep. The current can then be measured immediately before each potential change. Thus, the effect of the charging current could be minimized, achieving a higher sensitivity.

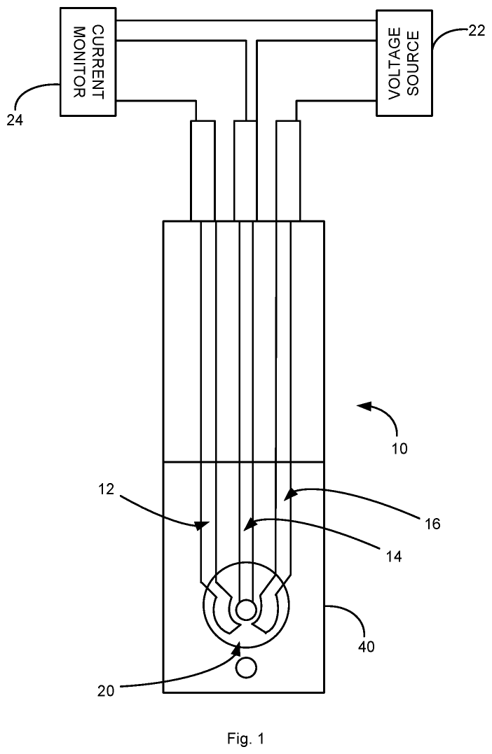

FIG. 1 illustrates a biosensor 10 in accordance with an embodiment of the application. The sensor 10 is a three-electrode sensor including a counter electrode 12, a working electrode 14, and a reference electrode 16 that are formed on a surface of a substrate. A dielectric layer 40 covers a portion of the working electrode 12, counter electrode 14 and reference electrode 16. The dielectric layer 40 includes an aperture 20 that defines a detection region of the working electrode 12, counter electrode 14, and reference electrode 16, which is exposed to samples containing an analyte to be detected. A bioconjugated anti-AMACR antibody and/or anti-PSA antibody (not shown) that is selective to AMACR and/or PSA is conjugated or linked to a surface of the exposed portion of the working electrode with a linker. The surface of the working electrode can be free of a self-assembled monolayer. The linked AMACR antibody and/or anti-PSA antibody can bind selectively to AMACR and/or PSA in the biological sample.

The biosensor can also include a voltage source 22 for applying a voltage potential to the working electrode, counter electrode, and/or reference electrode and a measuring device or current monitor 24 for measuring the current flow between the working electrode and counter electrode. The interaction of the AMACR antibody and/or anti-PSA antibody and the AMACR and/or PSA can be detected using electrochemical analytical techniques, such as cyclic voltammetry (CV), differential pulse voltammetry (DPV), to determine the presence of AMACR and/or PSA in the sample.

The working electrode 14 is poised at an appropriate electrochemical potential such that the current that flows through the electrode changes when the AMACR antibody and/or anti-PSA antibody binds to the AMACR and/or PSA in the sample. The function of the counter electrode 12 is to complete the circuit, allowing charge to flow through the sensor 10. The working electrode 14 and the counter electrode 12 are preferably formed of the same material, although this is not a requirement. Examples of materials that can be used for the working electrode 14 and counter electrode 12 include materials that can are reactive with the bioconjugated AMACR antibody and/or anti-PSA antibody to allow linking or conjugation of the anti-AMACR antibody and/or anti-PSA antibody to the surface of the working electrode. For example, the working electrode can be formed from gold, platinum, silver, palladium copper, alloys thereof, and composites thereof.

The AMACR antibody and/or anti-PSA antibody, which is conjugated or linked to the working electrode, can selectively bind to AMACR and/or PSA. In some embodiments, the AMACR antibody and/or anti-PSA antibody can include monoclonal and polyclonal antibodies, immunologically active fragments (e.g., Fab or (Fab)2 fragments), antibody heavy chains, humanized antibodies, antibody light chains, and chimeric antibodies. Antibodies, including monoclonal and polyclonal antibodies, fragments and chimeras, may be prepared using methods known in the art (see, for example, R. G. Mage and E. Lamoyi, in “Monoclonal Antibody Production Techniques and Applications”, 1987, Marcel Dekker, Inc.: New York, pp. 79-97; G. Kohler and C. Milstein, Nature, 1975, 256: 495-497; D. Kozbor et al., J. Immunol. Methods, 1985, 81: 31-42; and R. J. Cote et al., Proc. Natl. Acad. Sci. 1983, 80: 2026-203; R. A. Lerner, Nature, 1982, 299: 593-596; A. C. Nairn et al., Nature, 1982, 299: 734-736; A. J. Czernik et al., Methods Enzymol. 1991, 201: 264-283; A. J. Czernik et al., Neuromethods: Regulatory Protein Modification: Techniques & Protocols, 1997, 30: 219-250; A. J. Czemik et al., NeuroNeuroprotocols, 1995, 6: 56-61; H Zhang et al., J. Biol. Chern. 2002, 277: 39379-39387; S. L. Morrison et al., Proc. Natl. Acad. Sci., 1984, 81: 6851-6855; M. S. Neuberger et al., Nature, 1984, 312: 604-608; S. Takeda et al., Nature, 1985, 314: 452-454). AMACR antibody and/or anti-PSA antibodies to be used in the biosensor can be purified by methods well known in the art (see, for example, S. A. Minden, “Monoclonal Antibody Purification”, 1996, IBC Biomedical Library Series: Southbridge, Mass.). For example, anti-AMACR antibodies, and/or anti-PSA antibodies can be affinity purified by passage over a column to which a protein marker or fragment thereof is bound. The bound anti-AMACR antibodies, and/or anti-PSA antibodies can then be eluted from the column using a buffer with a high salt concentration. Anti-AMACR antibodies and/or anti-PSA antibodies, having binding affinities in the picomolar to micromolar range are suitable. Such interaction can be reversible. Instead of being prepared, anti-AMACR and/or anti-PSA antibodies to be used in the methods described herein may be obtained from scientific or commercial sources.

The anti-AMACR antibodies and/or anti-PSA antibodies are bioconjugated with a linker prior to linking or conjugating them to the surface of the working electrode. The linker can be a heterobifunctional linker of about 3 to about 10 atoms in length and include a first end and a second end. The first end can include an acyl group that is bound to a primary amine group of an amino acid residue of the antibody. The amine group can include, for example, a primary amine group of a lysine residue of the antibody. The second end can include a sulfhydryl group that is reactive with and can bind to a bare surface of the working electrode. The first end and the second end of the linker can be separated with a branched, unbranched, or straight aliphatic chain of about 1 to about 8 carbons in length, e.g., about 2 to about 7 carbon atoms in length or about 3 to about 6 atoms in length, to allow minimal distance between the antibody and the surface of the working electrode.

In some embodiments, the linker can include an N-hydroxysuccinimide ester that reacts with an amine group of a lysine residue the anti-AMACR antibody and/or anti-PSA antibody and a sulfhydryl group that reacts with the surface of the working electrode. The sulfhydryl group can also be coupled to a protecting group that is removed prior to reaction of the sulfhydryl group with the surface of the exposed portion of the working electrode. In some embodiments, the linker can include at least one of N-succinimidyl S-acetylthioacetate, N-succinimidyl S-acetylthiopropionate, or 2-iminothiolane*HCl (e.g., Traut's reagent).

Advantageously, it was found that conjugation of the heterobifunctional linker to the anti-AMACR antibody and/or anti-PSA antibody prior to conjugation of the linker to the working electrode can shorten the preparation process of the sensor, enhance coverage of the surface of working electrode, minimize pinhole effects and enhance the sensitivity of the sensor for AMACR and/or PSA.

In order to minimize any non-specific binding on the working electrode surface and blocking any open surface area of the working electrode at least one blocking agent can be applied to the surface of the working electrode once anti-AMACR antibodies and/or anti-PSA antibodies have been conjugated or linked to the working electrode. The blocking agent can enhance the reproducibility and sensitivity of the biosensor by minimizing non-specific interactions on the working electrode. In some embodiments, the blocking agent can include dithiothreitol or casein. The blocking agent can be applied to the surface of the working at an amount effective to minimize non-specific binding of proteins or other molecules on the surface of the working electrode.

A redox solution can be applied to the working electrode for determining the quantity of AMACR and/or PSA in the sample bound to the anti-AMACR antibody and/or anti-PSA antibody. The redox coupling solution can include a redox mediator, such as potassium ferrocyanide/potassium ferricyanide (K₃Fe(CN)₆/K₄Fe(CN)₆), that is provided at equimolar concentration in a PBS solution.

The voltage source 22 can apply a voltage potential to the working electrode 14 and reference and/or counter electrode 16, 12, depending on the design of the sensor 10. The current between the working electrode 14 and counter electrode 16 can be measured with the measuring device or meter 24. Such current is dependent on interaction of the analyte with the antibody on the working electrode.

The amount or level of current measured is proportional to the level or amount of AMACR and/or PSA in the biological sample. In some embodiments, where the sample is a bodily sample obtained from a subject, once the current level generated by the sample and redox solution tested with the sensor is determined, the level can be compared to a predetermined value or control value to provide information for diagnosing or monitoring of the cancer (e.g., prostate cancer) in a subject that is associated with the presence or absence of the AMACR and/or PSA.

In some embodiments, the current level generated by the sample obtained from the subject can be compared to a current level of a sample previously obtained from the subject, such as prior to administration of a therapeutic. Accordingly, the methods described herein can be used to measure the efficacy of a therapeutic regimen for the treatment of a cancer associated with the level of AMACR and/or PSA in a subject by comparing the current level obtained before and after a therapeutic regimen. Additionally, the methods described herein can be used to measure the progression of cancer associated with the presence or absence of AMACR and/or PSA in a subject by comparing the current level in a bodily sample obtained over a given time period, such as days, weeks, months, or years.

The current level generated by a sample obtained from a subject may also be compared to a predetermined value or control value to provide information for determining the severity or aggressiveness of cancer associated with AMACR and/or PSA levels in the subject. A predetermined value or control value can be based upon the current level in comparable samples obtained from a healthy or normal subject or the general population or from a select population of control subjects.

The predetermined value can take a variety of forms. The predetermined value can be a single cut-off value, such as a median or mean. The predetermined value can be established based upon comparative groups such as where the current level in one defined group is double the current level in another defined group. The predetermined value can be a range, for example, where the general subject population is divided equally (or unequally) into groups, or into quadrants, the lowest quadrant being subjects with the lowest current level, the highest quadrant being individuals with the highest current level. In an exemplary embodiment, two cutoff values are selected to minimize the rate of false positive and negative results.

The biosensor illustrated in FIG. 1 can be fabricated on a substrate formed from polyester or other electrically non-conductive material, such as other polymeric materials, alumina (Al₂O₃), ceramic based materials, glass or a semi-conductive substrate, such as silicon, silicon oxide and other covered substrates. Multiple sensor devices can thus be formed on a common substrate. As will be appreciated, variations in the geometry and size of the electrodes are contemplated.

The biosensor can be made using a thin film, thick film, and/or ink-jet printing technique, especially for the deposition of multiple electrodes on a substrate. The thin film process can include physical or chemical vapor deposition. Electrochemical sensors and thick film techniques for their fabrication are discussed in U.S. Pat. No. 4,571,292 to C. C. Liu et al., U.S. Pat. No. 4,655,880 to C. C. Liu, and co-pending application U.S. Ser. No. 09/466,865, which are incorporated by reference in their entirety.

In some embodiments, the working electrode, counter electrode, and reference electrode may be formed using laser ablation, a process which can produce elements with features that are less than one-thousandth of an inch. Laser ablation enables the precise definition of the working electrode, counter electrode, and reference electrode as well as electrical connecting leads and other features, which is required to reduce coefficient of variation and provide accurate measurements. Metalized films, such as Au, Pd, and Pt or any metal having similar electrochemical properties, that can be sputtered or coated on plastic substrates, such as PET or polycarbonate, or other dielectric material, can be irradiated using laser ablation to provide these features.

In one example, a gold film with a thickness of about 300 A to about 2000 A can be deposited by a sputtering technique resulting in very uniform layer that can be laser ablated to form the working and counter electrodes. The counter electrode can use other materials. However, for the simplicity of fabrication, using identical material for both working and counter electrodes will simplify the fabrication process providing the feasibility of producing both electrodes in a single processing step. An Ag/AgCl reference electrode, the insulation layer, and the electrical connecting parts can then be printed using thick-film screen printing techniques.

A heterobifunctional linker can be conjugated to the anti-AMACR antibody and/or anti-PSA antibody. By way of example, a heterobifunctional linker, such as N-succinimidyl S-acetylthioacetate or N-succinimidyl S-acetylthiopropionate, can be dissolved in DMSO. The heterobifunctional linker solution can then be mixed with a solution that includes the anti-AMACR antibody and/or anti-PSA antibody at a ratio of heterobifunctional linker and antibody of about 10:1, 15:1, 20:1, 25:1 or more to react the N-succinimidyl group with an amine of a lysine residue of the anti-AMACR antibody and/or anti-PSA antibody and conjugate the heterobifunctional linker to the antibody.

In some embodiments, where the heterobifunctional linker includes a protecting group, such as an acetyl group coupled to a sulfhydryl group, the protecting group can be removed prior to reaction of the sulfhydryl group with the surface of the exposed portion of the working electrode using a deacetylation process. The deacetylation process can employ, for example, a hydroxylamine solution that is applied to the antibody.

Following conjugation of the heterobifunctional linker to the anti-AMACR antibody and/or anti-PSA antibody and optionally removal of the protecting group, the antibody can be conjugated to surface of the working electrode. A chemical cleaning procedure can initially be applied to remove any oxides and particles on the working electrode surface to decrease the electrode charge transfer resistance. Typically, a sensor with the exposed working electrode can be immersed in an alkaline solution (e.g., 2M KOH solution), a first acidic solution (e.g., 0.05M H₂SO₄ solution), and a second acidic solution (e.g., 0.05M HNO₃ solution) different than the first acidic solution in sequence. The sensor can be immersed in DI water between immersion in each solution.

The linked or bioconjugated anti-AMACR antibody and/or anti-PSA antibody can then be applied to the surface of the working electrode using a micro-flow incubation system to link the anti-AMACR antibody and/or anti-PSA antibody to the working electrode surface. Continuous flow incubation process can maximize the surface coverage of the anti-AMACR antibody and/or anti-PSA antibody and enhance the homogeneity and reproducibility of the incubation results compared with static dropping incubation. The flow rate can be set at about 10 μL/min, 20 μL/min, 30 μL/min, 40 μL/min, 50 μL/min, 60 μL/min, 70 μL/min, 80 μL/min or more with a retention time effective to allow reaction of the sulfhydryl group of the linker with the working electrode surface.

Following addition and linking of the anti-AMACR antibody and/or anti-PSA antibody to the working electrode, the working electrode surface can be blocked using a blocking agent to minimize any non-specific molecule (e.g., protein) bonding on the electrode surface. This step will enhance the reproducibility and sensitivity of the biosensor. In some embodiments, DTT (Dithiothreitol), casein, and/or other blocking agents can be used to cover the open surface area of the working electrode and minimize any non-specific protein coverage.

In other embodiments, a plurality of biosensors can be provided on a surface of a substrate to provide a biosensor array. The biosensor array can be configured to detect the AMACR and/or PSA concentration changes occurring in proximity to the array. The biosensor array can include a plurality biosensors arranged in a plurality of rows and a plurality of columns. Each biosensor comprises a working electrode, a counter electrode, and a dielectric layer covering a portion of the working electrode and counter electrode and defining an aperture exposing other portions of the working electrode and counter electrode. Anti-AMACR antibodies and/or anti-PSA antibodies for AMACR and/or PSA can be linked or conjugated to the working electrode as described herein. The anti-AMACR antibodies and/or anti-PSA antibodies can be the same or different for each biosensor of the array and can bind selectively to the AMACR and/or PSA. For example, at least some sensors in the array can include anti-AMACR antibodies, while other sensors can include anti-PSA antibodies. The biosensors of the array can be configured to provide at least one output signal representing the presence and/or concentration of AMACR and/or PSA proximate to a surface of the array. For each column of the plurality of columns or for each row of the plurality of rows, the array further comprises column or row circuitry configured to provide voltage potentials to respective biosensors in the column or row. Each biosensor in the row or column can potentially detect a different AMACR and/or PSA and/or biased to detect different analytes.

### Example

Self-assembled monolayer (SAM) is a promising platform technology for biosensor applications. Typically, SAM is formed by an alkane-linked thiol molecule producing a gold-sulfur (Au—S) bond with the gold electrode surface of a biosensor. Then, activation of the terminal functional group followed, immobilizing antigen binding, such as antibody, aptamer, and specific receptor. The formation of the gold electrode elements, working and counter electrodes, of the biosensor could be accomplished by various techniques and in different dimensions. In this example, the gold electrode element was a thin gold film, 50 nm in thickness and was deposited by sputtering physical vapor deposition by a roll-to-roll cost effective manufacturing method, producing the biosensor relatively inexpensively and effectively. This biosensor was a three-electrode configuration, and the details of the configuration and the characterization of this biosensor have been reported elsewhere. For a comprehensive development of this biosensor, six different SAMs were studied, compared, and assessed in this research. These general preparing procedures of a commonly used biosensor were complex and required days for preparation and consumed excess chemicals. Furthermore, the biosensors using SAMs had relatively low sensitivity and poor reproducibility, because of common monolayer defects, such as pinholes, inhomogeneity of surface coverage, and others. Therefore, a new technique for the preparation of a biosensor was used in this example, and it was the bioconjugation mechanism.

The bioconjugation mechanism conjugates two or more molecules, forming a novel complex embracing the combined properties of its individual components. This method makes a zero-length linkage between the protein and electrode elements of the biosensor possible. Furthermore, this bioconjugation technique will shorten the preparation process, enhancing the coverage of the biosensor surface/minimizing the pinhole effect. Consequently, this will improve the practical clinical application. The interaction between antibody and antigen remained biorecognition mechanism in this research endeavor. In this example, anti-AMACR and anti-PSA were modified by the bioconjugation technique using N-succinimidyl S-acetylthioacetate (SATA) to conjugate the antibody. The final product of the conjugation reaction was a thiol group-linked AMACR antibody or PSA antibody, which directly linked with the thin gold film electrode element surfaces of the biosensor through incubation. After being modified by a thiol-linked antibody, fabrication of an AMACR or a PSA biosensor was completed. This single-step preparation took approximately one day including the incubation time for the preparation of the biosensor.

Thus, the combination of bioconjugation technique in preparation, microfabrication of a thin gold film-based biosensor prototype, and differential pulse voltammetry (DPV) measurement technique results in a single-use, cost effective, and highly sensitive and selective biosensor for the detection of the biomarkers of prostate cancer, AMACR and PSA, a very attractive and practical diagnostic tool for the screening application of prostate cancer.

### Materials

Anti-AMACR (Cat. no. HPA019527) was obtained from Sigma-Aldrich (St. Louis, Mo., USA), and AMACR (Cat. no. MBS428004) was obtained from MyBioSource (San Diego, Calif., USA). Anti-PSA (Cat. no. ab76113) and PSA peptide (Cat. no. ab41421) were obtained from Abcam (Cambridge, Mass., USA). PBS 1.0 M (pH 7.4), human serum (Cat. no. H3667), DL-dithiolthreitol solution (Cat. no. 43816), 3-mercaptopropionic acid (3-MPA) (Cat. no. M5801), 6-mercapto-1-hexanol (Cat. no. 451088), 11-mercaptoundecanoic acid (Cat. no. 450561), N-(3-dimethylaminopropyl)-N′-ethylcarbodiimide hydrochloride (EDC) (Cat. no. E1769), Nhydroxysuccinimide (NHS) (Cat. no. 130672), and Nhydroxysulfosuccinimide sodium salt (Cat. no. 56485) were purchased from Sigma-Aldrich (St. Louis, Mo., USA). SATA (Cat. #26102) and dimethyl sulfoxide (DMSO) (Cat. #BP231-1) were obtained from Thermo Fisher Scientific (Pittsburgh, Pa. USA). Ethylenediaminetetraacetic acid (EDTA) (Cat. EDS) and hydroxylamine (Cat. #255580) were obtained from Sigma-Aldrich (St. Louis, Mo., USA). Concentrated H₂SO₄ (95.0-98.0 w/w %) and concentrated HNO3 (70% w/w %) were received from Fisher Scientific (Pittsburgh, Pa., USA). Potassium hydroxide, K3Fe(CN)6, and K4Fe(CN)6 (Cat. no. P1767, P3289, and P3667) were obtained from Sigma-Aldrich (St. Louis, Mo., USA). All the chemicals were used without further purification. A CHI 660C (CH Instrument, Inc., Austin, Tex., USA) electrochemical workstation was used for DPV characterization.

### Experiments

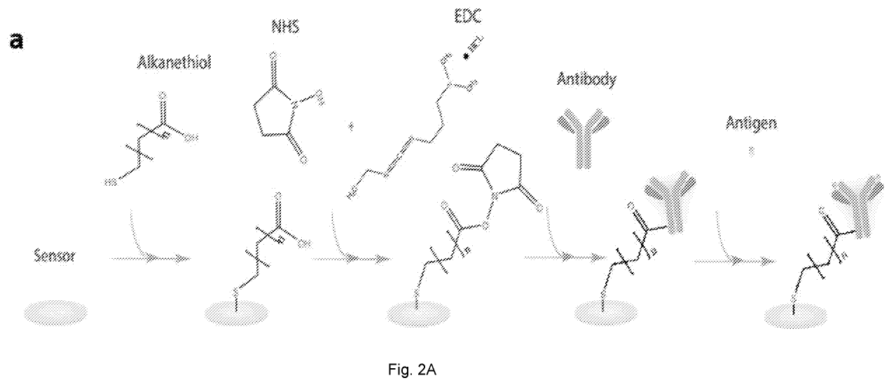

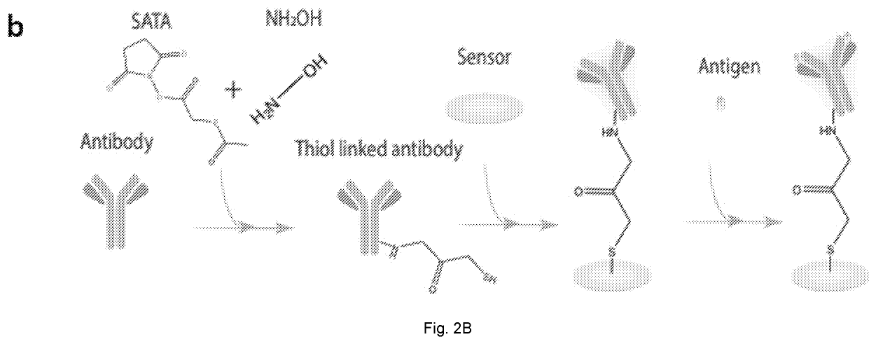

Preparation of an SAM-Based Biosensor. All the SAM systems were prepared in ethanol solution. Thin gold film-based biosensors were first cleaned as described in previous studies and immersed in the different SAM solutions for 24 h at room temperature. After 24 h of immersion in the SAM solution, the biosensors were rinsed by using deionized water and dried by using nitrogen gas. EDC (0.2 M) and 0.05 M NHS in 0.1 M PBS solution were prepared to activate the carboxylate group on the SAM by immersing the biosensors in the prepared SAM solution for 1 h at room temperature. Using anti-AMACR as an example, 20 μL of anti-AMACR solution with a concentration of 1 μg mL⁻¹ was drop-casted onto the biosensor after the activation process and incubated for 15 h at 4° C. The biosensor was then ready to be assessed for the effectiveness of the SAM system. The process of fabrication of the SAM biosensor is shown in FIG. 2A.

Preparation of the Thiol-Linked Anti-AMACR or Anti-PSA Protein. The bioconjugation mechanism was applied to create the thiol-linked anti-AMACR or anti-PSA protein. SATA was used to conjugate the antibody to produce a thiol-linked anti-AMACR or anti-PSA protein. Typically, for the preparation of the thiol-linked anti-AMACR, 0.5 mg of SATA was firstly dissolved in 1 mL of DMSO. The prepared SATA solution (1 μL) was mixed with 30 μL of anti-AMACR in 0.1 M PBS solution based on a molar ratio of 20:1 between SATA and the antibody. This mixed solution was incubated for 30 mM at room temperature. The solution was then diluted to a total volume of 500 μL by using 0.1 M PBS solution and transferred into an Amicon ultra-0.5 10 k filter tube, centrifuging at 12 000 rpm for 15 mM at 5° C. Twenty-five microliters of the filtered solution was obtained, and this filtered solution was reacted with 5 μL of 0.5 M hydroxylamine and 25 mM EDTA in 0.1 M PBS solution at room temperature for 2 h. An Amicon ultra-0.5 10 k filter tube was used again to filter out any molecules lower than 10 kDa molecular weight. This filtered solution was diluted to 500 μL by 10 mM EDTA in 0.1 M PBS solution and centrifuged at 12 000 rpm for 15 mM at 8° C. The solution was then diluted again using 10 mM EDTA in 0.1 M PBS solution to 500 μL and centrifuged again at 12 000 rpm for 15 mM at 8° C. After this second centrifuge process, the obtained solution was thiol-linked anti-AMACR. The bioconjugation process is shown in FIG. 2B. The thiol linked anti-PSA solution was prepared in a similar manner.

AMACR Biosensor and PSA Biosensor Fabrication Based on the Thiol-Linked Antibody. Using the anti-AMACR solution as an example, it reacted with the gold electrode surface, forming a strong Au—S bond linking the antibody onto the gold working electrode. The thiol-linked anti-AMACR was firstly diluted by 0.15 M NaCl and 10 mM EDTA in 0.1 M PBS solution to a concentration of 1.25 μg/mL. The gold biosensor was prepared in a batch of ten and cleaned. The diluted thiol linked anti-AMACR solution was vortexed. Twenty microliters of the solution at a concentration of 1.25 μg/mL was dropcasted onto the cleaned gold biosensor for incubation for 8 h at 4° C. The preparation step of the PSA biosensor is similar.

### Results

Evaluation of the Effectiveness of Coverage and Antibody Bonding Based on Different SAM Systems and Bioconjugated Systems. SAM is pivotal for binding the antibody. Different configurations of the SAM affect the orientation of the antibody and the electrode surface coverage of the biosensor, resulting in various binding effects and current signal outputs for electrochemical detection. Six different SAM systems were prepared examining their effectiveness in surface preparation of the biosensor in this study. The configurations of the six SAM systems are shown in Table 1. FIG. 2A shows the formation of the SAM system for biosensor development. The bioconjugation mechanism aims at the modification of an antibody to produce an external thiol linker on the antibody, which is able to directly react with the gold surface to form an Au—S bond without complex surface treatment. FIG. 2B shows the process of bioconjugation based biosensor fabrication. The detailed fabrication procedure for both processes is demonstrated in the Experiments section.

TABLE 1
Compositions of Six different SAM Systems^{a}

| SAM1 | SAM2 | SAM3 | SAM4 | SAM5 | SAM6 |
|---|---|---|---|---|---|
| 1.00 mM - | 1.00 mM | 5.00 mM | 1.00 mM | 1.00 mM | 3.00 mM |
| 3MPA | 3-MPA | MUA | MUA | MUA | MUA |
| | 0.13 mM | | 10.00 mM | 10.00 mM | 9.00 mM |
| | DTT | | MCH | 3-MPA | MCH |

^{a}3-MPA: 3:mercaptopropionic acid, MCH: 6-mercapto-1-hexanol, DTT: DL-dithiothreitol, and MUA: 11-mercaptoundecanoic acid

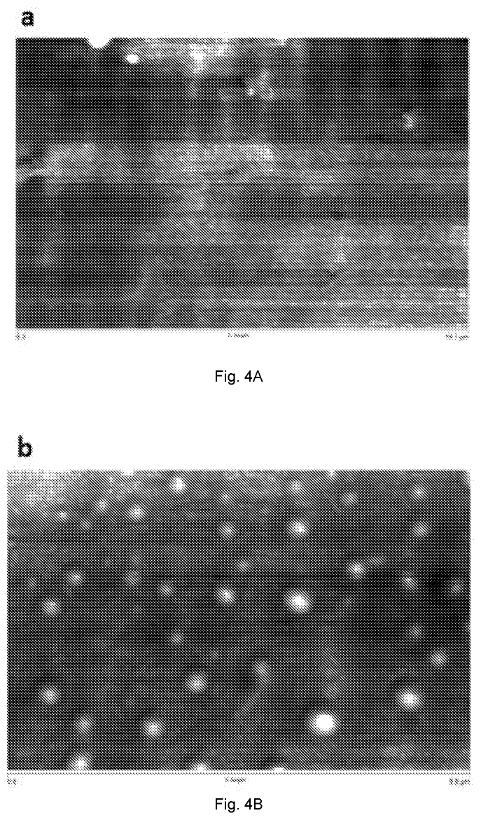

The sensitivity and the reproducibility of the biosensors prepared using different SAM systems and bioconjugation mechanism were evaluated. Three different AMACR antigen concentrations (50, 10, and 2 ng/mL) were prepared in 0.1 M phosphate-buffer saline (PBS) and drop-casted onto the biosensors with different SAM systems incubating for 2 h at room temperature. After incubation, the biosensor was rinsed by 0.1 M PBS solution and dried by nitrogen. DPV was used to measure the conductivity on the biosensor. A redox coupling solution (20 μL) of potassium ferrocyanide (K₄Fe(CN)₆) and potassium ferricyanide (K₃Fe(CN)₆) of 5 mM each was applied onto the biosensor surface. Of these six different SAM systems, SAM2- and SAM4-prepared biosensors showed unpromising reproducibility, and they were not investigated any further. FIG. 4A shows the sensitivity comparison for the other four SAM systems studied as described in Table 1. SAM3 demonstrated the best sensitivity; the difference among these four SAM modifications was minute and the sensitivity was at the level of 104 to 105 μA·μM⁻¹·cm⁻² which was only fair. The SAM1 system (3-MPA) retained the highest R-square value of these four SAM systems which was 0.698 (n=3). This R-square value was significantly lower than that of the bioconjugation-prepared biosensor [R-square=0.967 (n=5)], indicating a lower reproducibility of the SAM system for the detection of AMACR. The results of this study convincingly led us to apply the bioconjugation mechanism to prepare the biosensor for better achievement of an antibody-antigen recognition mechanism.

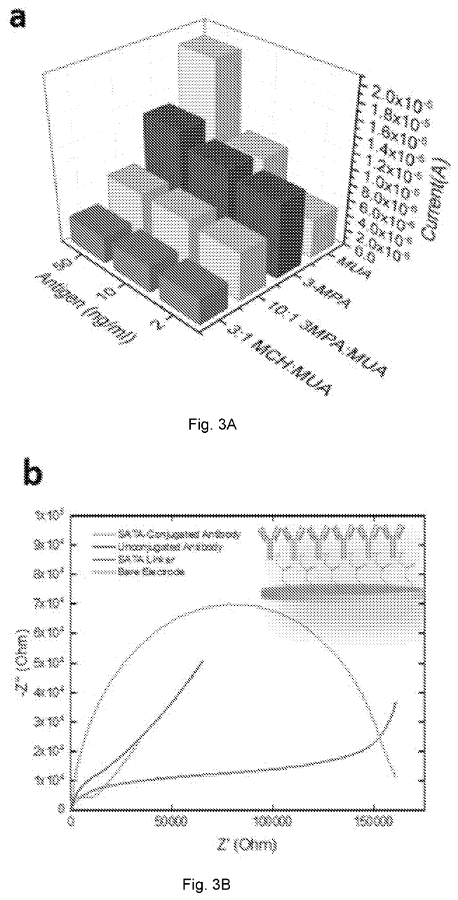

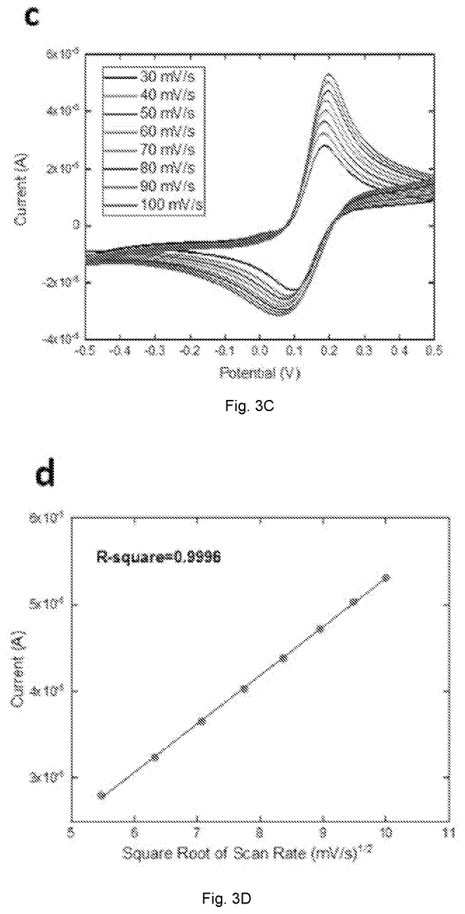

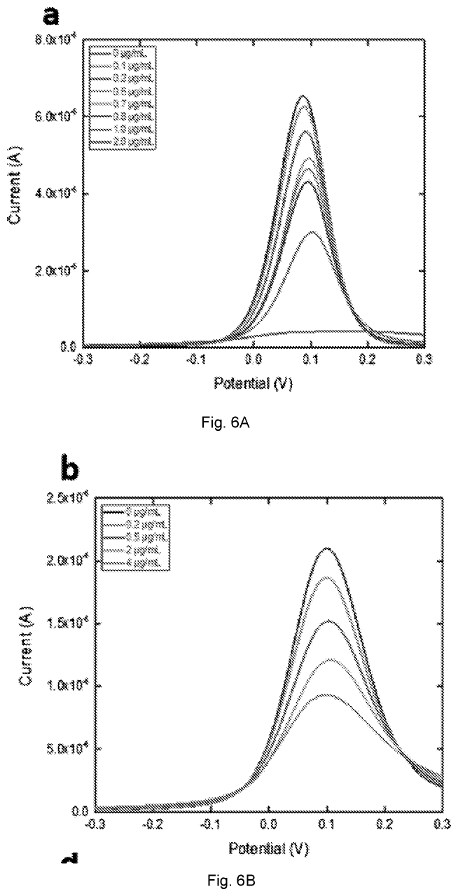

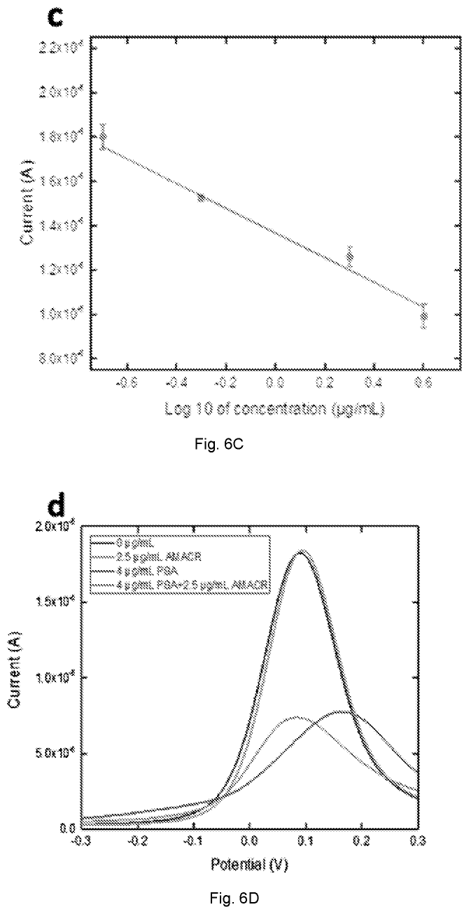

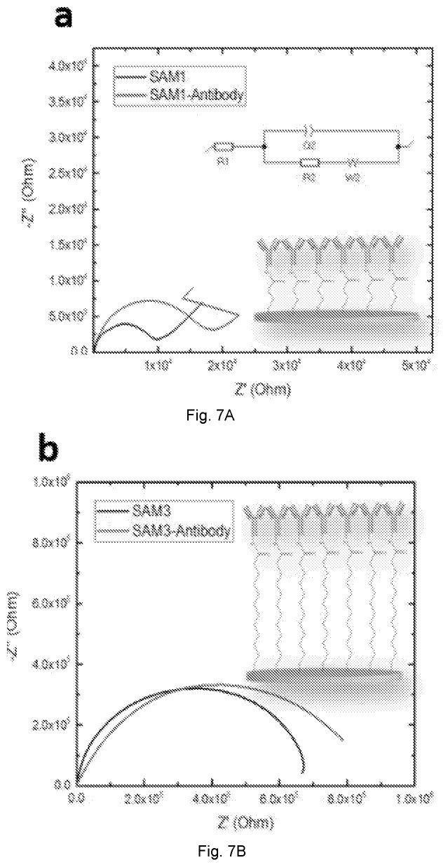

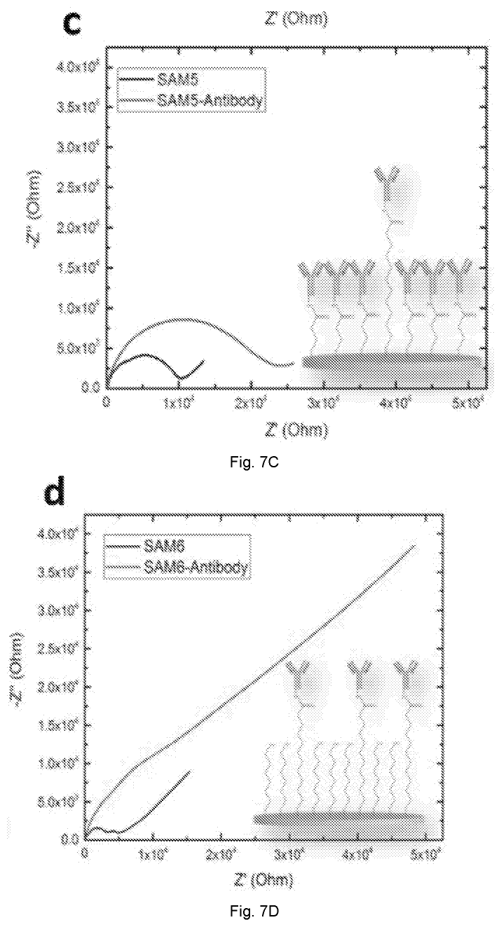

Electrochemical impedance spectroscopy (EIS) was used to assess the effectiveness of antibody binding by different SAM anti-AMACR protein. As the example, four selected SAM systems and the bioconjugated modified biosensors were investigated. The impedance difference between the SAM only biosensor and antibody-bonded SAM biosensor as well as that between the bare biosensor and the antibody-bonded bioconjugated biosensor was examined A concentration of 1.25 μg/mL anti-AMACR protein was used for all the SAM and bioconjugated systems. The biosensors were incubated for 15 h at 4° C. The biosensor was then rinsed by using 0.1 M PBS solution and dried by using nitrogen gas. The redox coupling solution (20 μL) of K₄Fe(CN)₆ and K₃Fe(CN)₆ of 5 mM of each was applied on the surface for the EIS measurement. The AC frequency range for the EIS measurement was 0.01-10 000 Hz. The Nyquist plots of this study are shown in FIG. 6 for four different SAMs and FIG. 3B for the bioconjugation-prepared antibody monolayer. The impedance difference before and after incubation of the antibody was calculated by EC-Lab software fitting the Randle circuit as shown in FIG. 6A, in which R1 represented the solution resistance, R2 characterized the charge transfer resistance, W2 indicated the diffusion limited process, and Q2 represented the electron transfer process. The difference of R2 value of each SAM system before/after incubation of the antibody was used to display the impedance difference as shown in Table 2.

TABLE 2
Resistance Value Difference Modeled by the Randle Circuit

| | Monolayer system | | | | |
|---|---|---|---|---|---|
| | SAM1 | SAM3 | SAM5 | SAM6 | Bioconjucation |
| Resistance | 21.3 | 116 | 51.1 | 552 | 6.64 × 10³ |
| difference (Ω) | | | | | |

The biggest impedance difference was shown from the bioconjugation method-prepared biosensor in FIG. 3B with a calculated resistance value at 6635Ω, which was significantly larger than that of any SAM system, indicating the maximum efficiency of antibody coverage by using bioconjugation for antibody monolayer formation. FIG. 3B shows the EIS characterization of the bioconjugation-prepared biosensor. The unconjugated antibody and SATA linker without antibody were also incubated for 2 h on the bare electrode. The changes of resistance on the sensor surface because of gold-protein affinity and only SATA linking with gold were not comparable with the resistance provided by the SATA-conjugated antibody, indicating that the best coverage of the electrode surface was produced by the SATA-conjugated antibody. High coverage of the antibody on the biosensor surface has been proved to be able to provide more sensitive quantification of analyte than lack of coverage condition.

To demonstrate the stability and reproducibility of the bioconjugation-prepared biosensor, after incubation with the bioconjugated antibody, cyclic voltammetry (CV) (FIG. 3C) was used to examine the biosensors in the presence of [Fe(CN)₆]³⁻⁴⁻ at different scan rates ranging from 30 to 100 mV/s. On the basis of the Randles-Sevcik equation, with a constant number of electrons transferred based on the redox event, fixed electrode surface area, diffusion coefficient, and the concentration of the redox coupling, the square root of the scan rate is linear proportional to the current outputs as shown in FIG. 3D, demonstrating good stability and reproducibility of the bioconjugated antibody-covered electrode based on the R-square value of 0.9996. Detailed costs for biosensor fabrication based on the SAM method and bioconjugation method were also compared and are shown in Table 3. The bioconjugation method-fabricated sensor was around $2.10/sensor and the SAM method-fabricated sensor was around 3.03/sensor, which outlays 44% more than the cost of the bioconjugation method.

TABLE 3
The cost comparison of bioconjugation fabricated
biosensor and SAM layer fabricated biosensor

| Bioconjugation | | | SAM layer | | |
|---|---|---|---|---|---|
| SATA | $40/100 | sensors | 11-MUA | $15/100 | sensors |
| Antibody | $40/100 | sensors | Ethanol | $17/100 | sensors |
| EDTA | $5/100 | sensors | NHS | $20/100 | sensors |
| Hydroxylamine | $5/100 | sensors | EDC | $91/100 | sensors |
| Sensor | $120/100 | sensors | Antibody | $40/100 | sensors |
| | | | Sensor | $120/100 | sensors |
| Total | $210/100 | sensors | Total | $303/100 | sensors |

| | | | |
|---|---|---|---|
| Cost/sensor | $2.10 | Cost/sensor | $3.03 |

Qualification of a Bioconjugation-Based Biosensor Surface Using Atomic Force Microscopy. Atomic force microscopy (AFM) was used to confirm the surface difference between a bare gold working electrode and a thiol linked AMACR antibody-covered gold working electrode. A scan size of 20 μm×10 μm was applied at a scan rate of 0.513 Hz. FIG. 4A,B shows the topography of a bare gold electrode image and a thiol-linked antibody-covered gold electrode image. FIG. 4A demonstrates a smooth gold electrode surface with a maximum height of 148 nm. FIG. 4B shows a more zigzag surface with a maximum height of 76 000 nm. The white plumped ball shapes indicate a rougher topography with the existence of the AMACR antibody. The white plumped balls also show a very similar size with radius around 200-250 nm, indicating a homogeneous distribution of the antibody on the surface. The qualification changes on the gold electrode surface observed by AFM provide solid proof of the capability of the bioconjugation mechanism on biosensor antibody film fabrication.

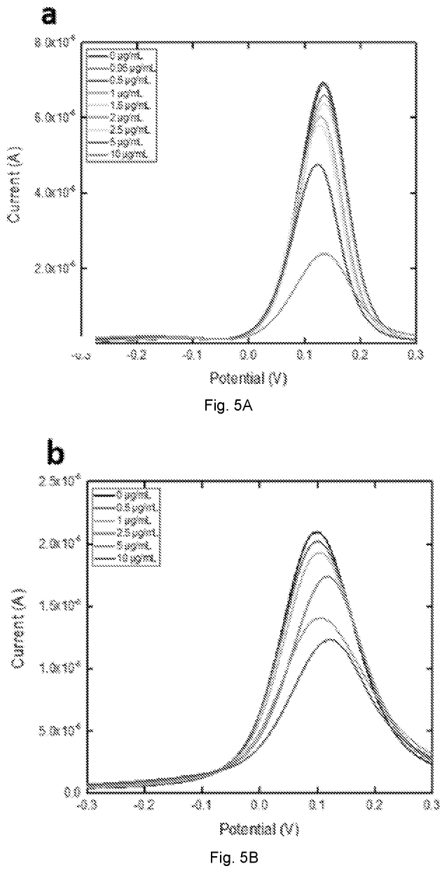

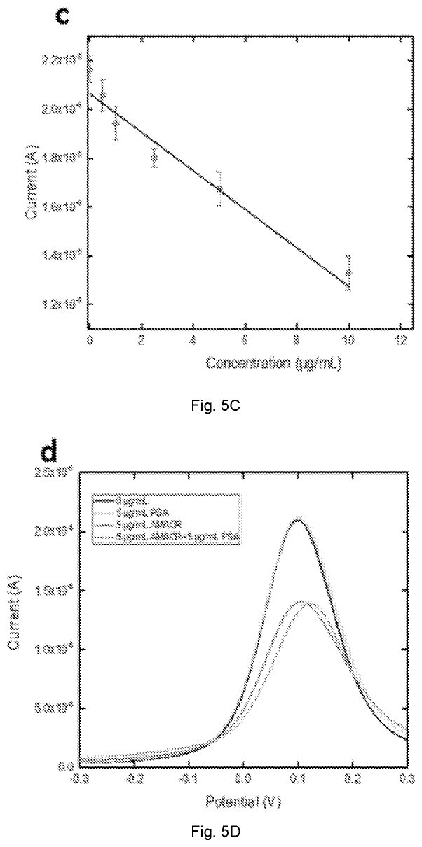

DPV Measurement of the AMACR Antigen in PBS and Undiluted Human Serum. The bioconjugation method-prepared AMACR biosensor was then used for the detection of an antigen of AMACR of different concentrations. Eight different concentrations of AMACR antigen were prepared in 0.1M PBS solution ranging from 10 to 0.05 μg/mL. The antigen sample (20 μL) was placed onto the AMACR biosensor and was incubated for 1 h at room temperature. The AMACR biosensor was then rinsed by 0.1 M PBS and dried by nitrogen gas. A redox probe solution (20 μL) of K₄Fe(CN)₆ and K₃Fe(CN)₆ of 5 mM each was applied onto the biosensor surface, and DPV measurement was then made. DPV was conducted at the potential range from −0.3 to 0.3 V. FIG. 5A shows the DPV measurement of eight different concentrations with a limitation of testing found at 0.05 μg/mL. The same experiment was also conducted using the AMACR antigen in undiluted human serum with a limitation of detection as shown in FIG. 5B, and the calibration is shown in FIG. 5C with a linear fit of Y=2.30×10⁻⁵ X=6.39×10⁻⁶ and Rsquare value of 0.900 (n=5).

DPV Measurements of PSA in PBS and Undiluted Human Serum. For comprehensive detection of prostate cancer, PSA was also evaluated using the bioconjugation mechanism-prepared PSA biosensor and DPV. PSA antigen in PBS solution was firstly tested based on an antibody concentration of 0.27 μg/mL. Concentrations of PSA antigen ranging from 2 to 0.1 μg/mL were prepared in 0.1 M PBS solution. The selected PSA antigen solution (20 μL) was dropcasted on the prepared PSA biosensor and incubated for 1 h at room temperature. The PSA biosensor was then rinsed by 1 mL of 0.1 M PBS solution and dried by nitrogen gas. A redox probe solution (20 μL) of K₄Fe(CN)₆ and K₃Fe(CN)₆ of 5 mM each was applied onto the biosensor surface, and DPV measurement was then made. DPV measurement is shown in FIG. 6A with a detection limit of 0.1 μg/mL. DPV measurement on PSA in undiluted human serum was also conducted with a PSA antigen concentration range of 0-4 μg/mL using an antibody concentration of 0.55 μg/mL. The same procedure as described in the PBS test was applied for the undiluted human serum test. DPV measurement is shown in FIG. 5B with a detection limit of 0.2 μg/mL, and its calibration curve is shown in FIG. 5C with a linear fit of Y=2.41×10⁻⁵ X=4.33×10⁻⁷ and R-square value of 0.967 (n=5).

Interference Study of the AMACR Biosensor and PSA Biosensor. Interference studies on the AMACR biosensor and PSA biosensor were conducted to confirm the selectivity of each biosensor. For the AMACR biosensor study, undiluted human serum with a PSA antigen concentration of 5 μg/mL was mixed with 5 μg/mL of AMACR antigen. The same incubation and detection procedures were conducted. The DPV measurement result is shown in FIG. 5D, in which the serum with mixed PSA/AMACR antigens showed no current output difference with only AMACR antigen in the serum, indicating that the PSA antigen did not interfere with the AMACR measurement by the AMACR biosensor. Similarly, for the PSA biosensor study, undiluted human serum with an AMACR antigen concentration of 2.5 μg/mL was mixed with 4 μg/mL PSA antigen solution. The same incubation and detection procedures were conducted. The DPV measurement result is shown in FIG. 6D, in which the mixed PSA/AMACR antigens showed no current output difference with only the PSA antigen in the serum, indicating that the AMACR antigen did not interfere with the PSA measurement by the PSA biosensor.

Bioconjugation mechanism has shown promising ability in applications for biosensor development. The fabrication process using the bioconjugation mechanism demonstrated the advantages of time-efficiency, cost-effectiveness, and most importantly, a simple method that makes it possible for medical professionals to operate the biosensor fabrication process. Two different biosensors for the detection of prostate cancer were fabricated. The developed biosensors showed excellent capability in the detection of biomarkers of prostate cancer in both PBS and undiluted human serum with good selectivity proved by two different interference studies.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1. A system for detecting AMACR and/or PSA in a biological sample, the system comprising:
a sensor that includes a substrate, a working electrode formed on a surface of the substrate; a counter electrode formed on the surface of the substrate; a dielectric layer covering a portion of the working electrode and counter electrode and defining an aperture exposing other portions of the working electrode and counter electrode; and a plurality of bioconjugated anti-AMACR antibodies and/or anti-PSA antibodies that are selective to AMACR or PSA, respectively, each of the bioconjugated anti-AMACR antibodies and/or anti-PSA antibodies including a linker that conjugates the bioconjugated anti-AMACR antibodies and/or anti-PSA antibodies to a surface of the exposed portion of the working electrode, the linker being about 3 to about 6 atoms in length and including a first end and a second end, the first end including an acyl group that is bound to a lysine group of the antibody and the second end including a sulfhydryl group that is bound to the surface of the working electrode;
a redox solution that is applied to the working electrode for determining the quantity of AMACR and/or PSA in the sample bound, respectively, to the anti-AMACR antibodies and/or anti-PSA antibodies, and
a measuring device for applying voltage potentials to the working electrode and counter electrode and measuring the current flow between the working electrode and counter electrode.

2. The system of claim 1, wherein the surface of the working electrode is free of a self-assembled monolayer.

3. The system of claim 1, wherein the working electrode and the counter electrode comprise metalized films.

4. The system of claim 3, wherein the working electrode and counter electrode independently comprise gold, platinum, palladium, silver, alloys thereof, and composites thereof.

5. The system of claim 3, wherein the metalized films are provided on the surface of the substrate by sputtering or coating the films on the surface and wherein the working electrode and the counter electrode are formed using laser ablation to define the dimensions of the working electrode and the counter electrode.

6. The system of claim 1, wherein the redox solution comprises potassium ferrocyanide/potassium ferricyanide solution.

7. The system of claim 1, further comprising a reference electrode on the surface of the substrate, the dielectric covering a portion of the reference electrode.

8. The system of claim 1, wherein linker includes an N-hydroxysuccinimide ester that reacts with an amine group of a lysine residue and a sulfhydryl group that reacts with the surface of the working electrode.

9. The system of claim 1, wherein the sulfhydryl group of the linker is coupled to a protecting group the protecting group being removed prior to reaction of the sulfhydryl group with the surface of the exposed portion of the working electrode.

10. The method of claim 8, wherein the linker includes at least one of N-succinimidyl S-acetylthioacetate or N-succinimidyl S-acetylthiopropionate.

11. The system of claim 1, wherein the sample comprises blood or serum.

12. A system for detecting prostate cancer in a subject, the system comprising:
a sensor that includes a substrate, a working electrode formed on a surface of the substrate; a counter electrode formed on the surface of the substrate; a dielectric layer covering a portion of the working electrode and counter electrode and defining an aperture exposing other portions of the working electrode and counter electrode; and a plurality of bioconjugated anti-AMACR antibodies and/or anti-PSA antibodies that are selective to AMACR or PSA, respectively, each of the bioconjugated anti-AMACR antibodies and/or anti-PSA antibodies including a linker that conjugates the bioconjugated anti-AMACR antibodies and/or anti-PSA antibodies to a surface of the exposed portion of the working electrode, the linker being about 3 to about 6 atoms in length and including a first end and a second end, the first end including an acyl group that is bound to a lysine group of the antibody and the second end including a sulfhydryl group that is bound to the surface of the working electrode;
a redox solution that is applied to the working electrode for determining the quantity of AMACR and/or PSA in the sample bound, respectively, to the anti-AMACR antibodies and/or anti-PSA antibodies, and
a measuring device for applying voltage potentials to the working electrode and counter electrode and measuring the current flow between the working electrode and counter electrode.

13. The system of claim 12, wherein the surface of the working electrode is free of a self-assembled monolayer.

14. The system of claim 12, wherein the working electrode and the counter electrode comprise metalized films.

15. The system of claim 14, wherein the working electrode and counter electrode independently comprise gold, platinum, palladium, silver, alloys thereof, and composites thereof.

16. The system of claim 14, wherein the metalized films are provided on the surface of the substrate by sputtering or coating the films on the surface and wherein the working electrode and the counter electrode are formed using laser ablation to define the dimensions of the working electrode and the counter electrode.

17. The system of claim 12, wherein the redox solution comprises potassium ferrocyanide/potassium ferricyanide solution.

18. The system of claim 12, further comprising a reference electrode on the surface of the substrate, the dielectric covering a portion of the reference electrode.

19. The system of claim 12, wherein linker includes an N-hydroxysuccinimide ester that reacts with an amine group of a lysine residue and a sulfhydryl group that reacts with the surface of the working electrode.

20. The system of claim 12, wherein the sulfhydryl group of the linker is coupled to a protecting group the protecting group being removed prior to reaction of the sulfhydryl group with the surface of the exposed portion of the working electrode.

21. The method of claim 19, wherein the linker includes at least one of N-succinimidyl S-acetylthioacetate or N-succinimidyl S-acetylthiopropionate.

22. The system of claim 12, wherein the sample comprises blood or serum.

23-33. (canceled)
